(19) United States
(12) Patent Application Publication
(10) Pub. No.: US 2024/0337670 A1
(43) Pub. Date: Oct. 10, 2024

(54) Divider for Separating Lipoproteins from Blood and Related Systems and Methods of Use

(71) Applicant: HEART, Health Resource Technology, LLC, Houston, TX (US)

(72) Inventors: Chu-Huang Mendel Chen, Houston, TX (US); Ming-Hsien Tsai, Houston, TX (US)

(21) Appl. No.: 18/297,423

(22) Filed: Apr. 7, 2023

(51) Int. Cl. G01N 33/92; B01L 3/00; G01N 1/34

(52) U.S. Cl. CPC G01N 33/92; B01L 3/502761; G01N 1/34; B01L 2300/0636; B01L 2300/0681; B01L 2300/0874; B01L 2300/0883; B01L 2400/0487

(57) ABSTRACT

Devices, systems, and processes are disclosed herein for separating a substance from a sample using size-exclusion chromatography (SEC) in a compact and configurable apparatus (referred to as a divider or separator). The divider separates the substance using SEC by having the sample flow through a conduit that has a length greater than the distance between an inlet and an outlet of the divider.

### FIELD

Implementations of the present disclosure relate to devices and methods for dividing or separating lipoproteins from blood.

### BACKGROUND

Blood includes plasma, which includes lipoproteins. Lipoproteins are important samples for indicating cardiovascular and other health conditions. Lipoproteins are often measured by performing a blood test, by drawing blood samples from a subject (e.g., a human patient) and analyzing the blood samples using various methods in laboratories. For example, conventional methods include separating the lipids (e.g., cholesterol, triglycerides, and lipoproteins) from other components of the blood using centrifugation. That is, the blood sample is spun at high speeds for a long duration for the lipids of various densities to settle at different locations.

The centrifugation process may require relatively a long period of time, for example as compared to the analysis time required for the separated lipids. For example, the length of time for separating the lipids may vary but usually takes several minutes when the spinning speeds are at thousands of rounds per minute. The time requirement may also depend on sample size/volume, the specific test objectives, and other factors. As such, lipid separation using centrifugation processes is time consuming and could induce inaccuracies due to different factors.

### SUMMARY

The present disclosure provides devices, systems, and processes for separating a substance from a sample using size-exclusion chromatography (SEC, also known as gel-filtration chromatography) in a compact and configurable apparatus (referred to as a divider or separator). The divider separates the substance using SEC by having the sample flow through a conduit that has a length greater than the distance between an inlet and an outlet of the divider, unlike conventional SEC columns that have respective inlets and outlets separated at distances no less than the conduit length.

In a first general aspect, a divider of this disclosure may receive the sample at an inlet affixed onto a housing of the divider. The divider includes a conduit contained in the housing. The conduit is coupled to the inlet and is filled with a composite resin, such as agarose, dextran, among others, for separating the substance from the sample. The divider includes an outlet affixed onto the housing and coupled to the conduit. The separated substance exits the outlet and is then measured or inspected. The distance between the outlet and the inlet is less than a total length of the conduit.

In some embodiments, the conduit includes a plurality of parallel tubes. Each of the parallel tubes has a coupler (e.g., a cap) at each end for connecting two or more of the parallel tubes in series. For example, the coupler includes a fitting and a flexible tubing. The fitting sealingly connects an open end of the flexible tubing to a corresponding one of the parallel tubes, enabling the flexible tubing to carry pressurized fluids from one of the parallel tubes to another. The fitting may include a thread, a seal, and a handle for installation and removal. In some cases, the divider further includes a cap to enclose the coupler and protect the flexible tubing.

In some embodiments, the resin composite includes a cross-linked agarose. The test sample includes plasma, such that when the plasma is pumped through the conduit, a lipoprotein is separated from the plasma by the cross-linked agarose. An absorbance value of the separated lipoprotein in the plasma is measurable in an ultraviolet (UV) detector (or other spectrometers that measure the absorbance or fluorescence of the lipoprotein). In some cases, the cross-linked agarose has a particle size between 8 and 30 microns. The test sample may have a volume ranged between 50 and 200 micro-liters.

In another general aspect, a system is disclosed herein for substance separation using SEC with a separator. The system includes a liquid chromatography pump, a liquid chromatography UV detector, and the separator therebetween. During operation, the liquid chromatography pump powers a test sample through the separator to separate a substance in the test sample. The liquid chromatography UV detector then receives the substance and measures the absorbance or fluorescence of the substance. The separator includes an inlet affixed onto a housing. The inlet receives a test sample pumped from the liquid chromatography pump. The separator further includes a conduit contained in the housing. The conduit is coupled to the inlet for receiving the test sample for SEC separation using a resin composite filling the conduit. The separator further includes an outlet affixed onto the housing and coupled to the conduit, such that a distance between the outlet and the inlet is less than a total length of the conduit. The outlet then provides samples separated from the test samples by the separator to the liquid chromatography UV detector.

In another general aspect, a method is disclosed herein for substance separation using SEC. The method includes providing a test sample using a liquid chromatography pump to an inlet of a divider. The method further includes separating a substance from the test sample in a conduit contained in a housing of the divider. The conduit is coupled to the inlet for receiving the test sample for SEC separation using a resin composite filling the conduit. The method further includes providing the substance separated from the test samples by the resin composite to a liquid chromatography ultra-violet (UV) detector via an outlet of the divider. The outlet is affixed onto the housing and coupled to the conduit. The distance between the outlet and the inlet is less than the total length of the conduit.

Various examples are described in detail below.

### BRIEF DESCRIPTION OF THE DRAWINGS

The described embodiments and the advantages thereof may best be understood by reference to the following description taken in conjunction with the accompanying drawings. These drawings in no way limit any changes in form and detail that may be made to the described embodiments by one skilled in the art without departing from the spirit and scope of the described embodiments.

FIG. 1 illustrates an example configuration for liquid chromatography using a divider of the present disclosure.

FIG. 2 illustrates a perspective exploded view of an example divider, in accordance with certain aspects of the present disclosure.

FIG. 3 illustrates an example front view of the divider of FIG. 2, in accordance with certain aspects of the present disclosure.

FIG. 4 illustrates an example perspective view and a local view of the divider of FIG. 2, in accordance with certain aspects of the present disclosure.

FIG. 5 illustrates an example cap, its perspective view and cross sectional view for use with the divider of FIG. 2, in accordance with certain aspects of the present disclosure.

FIG. 6 illustrates fittings and tubes for use with the divider of FIG. 2, in accordance with certain aspects of the present disclosure.

FIG. 7 illustrates configuration variations of inlets, outlets, and conduit lengths of the divider of FIG. 2, in accordance with certain aspects of the present disclosure.

FIG. 8 illustrates a flow diagram of methods of system performance using the divider of FIG. 2, in accordance with certain aspects of the present disclosure.

FIG. 9 illustrates example measurement results, in accordance with certain aspects of the present disclosure.

Like numerals indicate like elements.

### DETAILED DESCRIPTION

The present disclosure provides devices, systems, and processes for separating a substance from a sample using size-exclusion chromatography (SEC, also known as gel-filtration chromatography) in a compact and configurable apparatus (referred to as a divider or separator). For example, the divider may separate lipids from plasma for detection and analysis. The disclosed divider enables rapid processing of a large number of blood samples and provides for high resolution results in a compact form factor.

Conventionally, centrifugation is used to obtain blood plasma, which includes various proteins, lipids, and other molecules indicative of human health. Centrifugation makes use of the different densities of the proteins and lipids (e.g., cholesterol, triglycerides, and lipoproteins) for separation. The length of time to separate the lipids in a blood sample using centrifugation can vary and usually takes several minutes (depending on spinning speeds, sample sizes, and other factors, such as temperatures, container shapes, etc.). After separation, the lipid samples may further require processing and quantification measurements, which may take a long period of time.

The disclosed divider overcomes the drawbacks of centrifugation by using SEC to quickly separate lipoproteins from the blood sample. For example, when a pump powers a blood sample into an inlet of the divider and through a conduit, the lipoproteins of the blood sample exit from the outlet of the divider. By using proper conduit size and SEC polymers, the separation and detection processes may be much faster than centrifugation methods. Furthermore, less volume of the blood sample may be needed using SEC than the volume needed using centrifugation methods for results of similar accuracy (this speeds up the blood drawing processes and improves patient experience).

Existing SEC configurations often include large and lengthy tubes connected in series. As such, the connected tubes often occupy a large space and are inconvenient for transportation or replacement (and thus impractical for use in clinics, hospitals, or the like). Furthermore, known SEC configuration may not be able to separate lipoproteins from blood samples because the materials (e.g., polymers) used in the stationary phase may not prevent other proteins from passing through, lowering the separation effectiveness and reducing the detection/analysis accuracies. The present disclosure overcomes these issues by packing the SEC polymers in a zig-zag shaped, length-reconfigurable conduit so that only the substance of smaller than a target size may pass through and exit the outlet for analysis.

An example device has a configurable, replaceable, and compact form factor that allows for quick connection with common liquid chromatography instruments, such as liquid chromatography pumps and ultra-violet (UV) detectors, such that a large number of samples may be tested quickly without waiting through traditional centrifugation periods. The example device employs SEC or ion-exchange chromatography (IEC), and uses cross-linked agarose as sample fillers and blood protein as sample analytes. The disclosed devices and methods may be used in biomedical testing and lipoprotein purification. In an example, the disclosed methods improve chromatographic analysis resolution using an improved flow path to effectively reduce the installation space while providing a conduit having a total length no less than that of the conventional configurations.

At a high level, liquid chromatography is a separation technique used in chemistry, biochemistry, and other fields to separate and purify compounds. In principle, a sample mixture is dissolved in a liquid (the mobile phase) and then passed through a stationary phase (e.g., a medium or a column). The components of the sample interact with the stationary phase to different degrees and are separated as they pass through the stationary phase. In SEC, the stationary phase contains porous beads that allow smaller molecules to enter the pores, while larger molecules are excluded and elute more quickly.

Other examples of liquid chromatography include high-performance liquid chromatography, which uses a solid material packed into a column as the stationary phase when the mobile phase is a liquid solvent pumped through the column at high pressure. Another type of liquid chromatography includes ion exchange chromatography, which uses, in stationary phase, charged groups that attract or repel charged species in the sample. Another type of liquid chromatography includes affinity chromatography, in which the stationary phase contains a ligand that specifically binds to a target molecule in the sample. Although the present disclosure uses SEC as an example, the divider and techniques disclosed may similarly used in other types of liquid chromatography.

Basic steps of the liquid chromatography are as follows. First, sample is prepared by being dissolved in a suitable solvent and filtered to remove solid or particulate matter. Then, a small volume of the sample is injected into and through the stationary phase (e.g., a column). As the sample passes through the column, the individual components are separated. As the separated components elute from the column, a detector, such as an ultraviolet visualization spectrophotometer, measures the absorbance or fluorescence of the separated components. The data obtained from the detector may then be analyzed to determine the identity and quantity of the individual components in the sample.

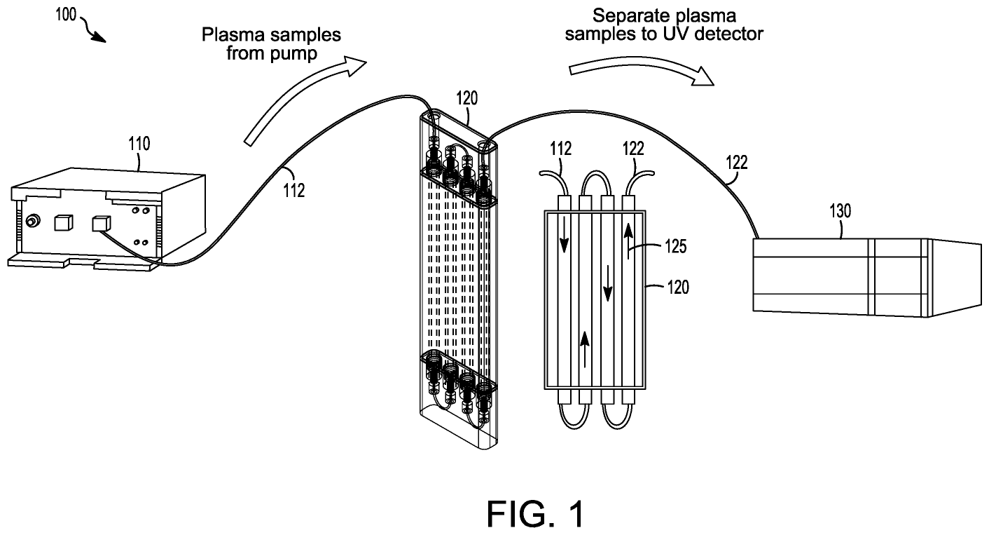

FIG. 1 illustrates an example configuration 100 for liquid chromatography using a divider of the present disclosure. As shown, a pump 110 transfers a sample 112 into a divider 120. The sample 112 passes through the stationary phase 125 of the divider and exits the outlet (as a separated portion 122 of the sample 112, also referred to as analytes or eluent herein). For example, the sample 112 may include blood plasma (e.g., whole or diluted) and the separated portion 122 may include lipids (e.g., cholesterol, triglycerides, and/or lipoproteins). The separated portion 122 is then provided to a detector 130. The detector 130 may include a UV detector for detecting and quantifying analytes, as molecules may absorb UV lights ranging between 190 and 400 nm wavelengths.

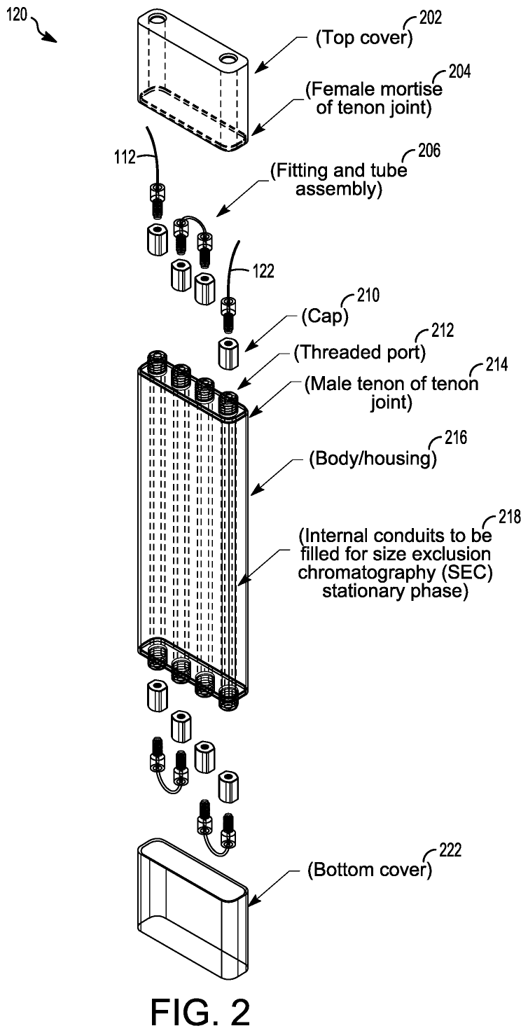

In general, as shown in the example configuration 100, a liquid chromatography system using SEC includes the pump 110 configured to deliver a mobile phase of the sample 112 through the divider 120 (functioning as a chromatography column) at certain flow rate(s). The detector 130 is positioned downstream from the divider 120 and configured to detect UV radiation (e.g., absorption, reflection, or fluorescence) from the eluent 122 exiting the divider 120. In some cases, the pump 110 and the detector 130 are in communication with a control system (not shown) configured to regulate the flow rate of the mobile phase delivered by the pump 110 and to receive signals from the detector indicative 130 of the presence and concentration of analytes in the eluent 122. Detailed description of the divider 120 is provided below in relation to FIGS. 2-7, after the following brief discussion on the pump 110 and the detector 130.

In some embodiments, the pump 110 may include a high-pressure pump capable of generating high pressures as well as accurate and precise flow rates. The pump 110 may deliver the mobile phase (the liquid that carries the sample through the divider 120) through the stationary phase 125 of the divider 120 at a constant flow rate. The flow rate may be critical for achieving reproducible separations. Examples of the pump 110 include syringe pumps, piston pumps, binary pumps, quaternary pumps, among others.

For example, syringe pumps use a motorized syringe to generate a pressure to deliver the mobile phase at a constant flow rate through the divider 120. Piston pumps use a reciprocating piston and may be more reliable than syringe pumps due to different materials being used at the piston-cylinder interface. As a result, piston pumps may also generate higher pressures than syringe pumps. Peristaltic pumps use a set of rollers to squeeze and release a flexible tubing and may achieve being gentle on large volumes of sample that need to be separated. Binary or quaternary pumps use multiple pumps to deliver multiple different mobile phases at different flow rates, which allows for the formation of a gradient elution. In an example, the pump 110 includes a piston pump for high pressure and reliable operations.

In some embodiments, the detector 130 measures the absorbance of the eluent (e.g., the separated portion 112) exiting the divider 120. In UV detection, a UV lamp is used to produce UV light that passes through the eluent and/or the sample molecules. The sample molecules absorb some of the UV light that causes a decrease in the intensity of the light that reaches the detector. The decrease in light intensity is proportional to the concentration of the analyte, allowing its concentration to be measured. In some cases, a specific wavelength (or a specific range) of the UV light is used to measure specific analyte. In some cases, fluorescence, reflection, or other light response or reactions may be used instead of absorbance for identifying and quantifying the substance being measured. The detector 130 may detect a wide range of samples, including proteins, nucleic acids, and small molecules.

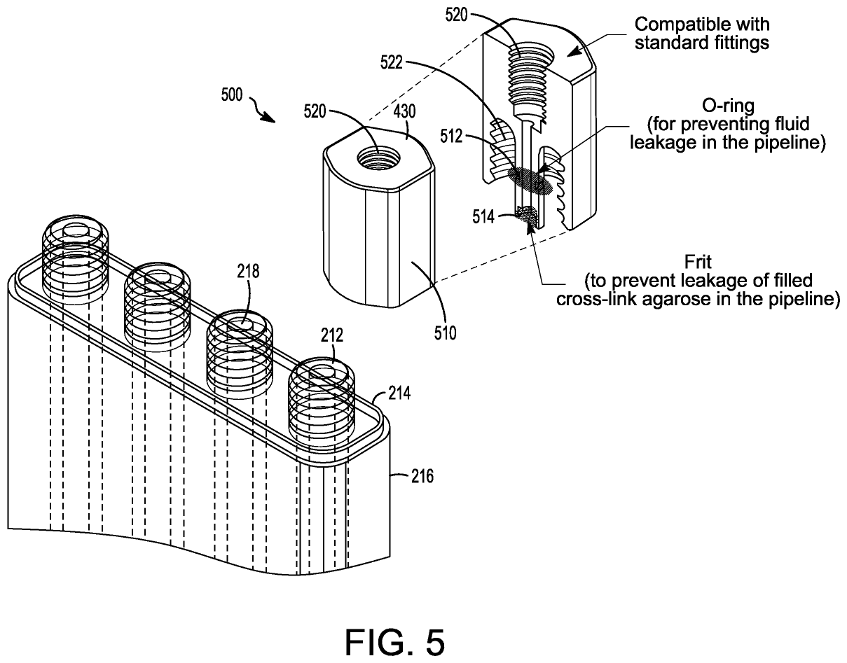

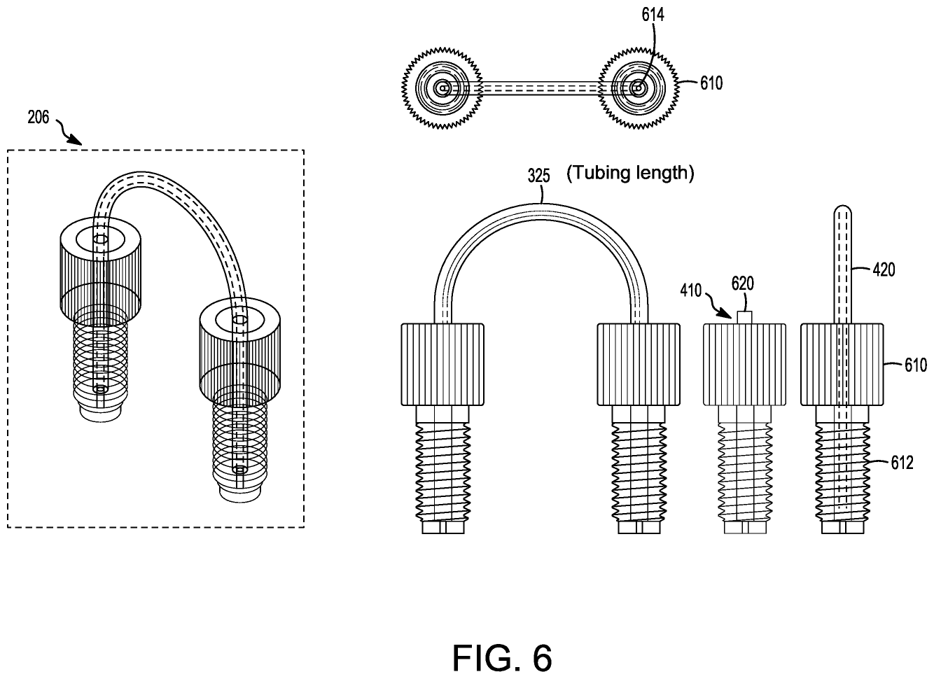

FIG. 2 illustrates a perspective exploded view of the example divider 120, in accordance with certain aspects of the present disclosure. As shown, the divider includes a top cover 202, a body (or housing, interchangeable herein) 216, and a bottom cover 222. The top cover 202 may include holes for connection tubes or connectors to reach the internal conduits 218 in the body 216. The top cover 202 and the bottom cover 222 may have female mortise mounts 204 for coupling with corresponding male tenon mounts 214 on the body 216. The top cover 202 and the bottom cover 222 protect the fitting and tub assembly 206 that configures the conduits 218 of the body 216. In some cases, each conduit 218 may have threaded ports 212 at respective ends for connecting with the fitting and tube assembly 206 via corresponding caps 210. Details of the structures of the caps 210 and fitting and tube assembly 206 are illustrated in FIG. 5 and FIG. 6 respectively.

The tenon joint 204 and 214 use tenon fittings to connect the top cover 202 or the bottom cover 222 to the body 216, creating a leak-free protection housing for the fitting and tube assembly 206. The tenon joint includes the male tenon 214, which is a protrusion on one end of the body or cover, and a female mortise 204, which is a corresponding indentation or hole in the other piece. The tenon 214 and mortise 204 are to fit together tightly, creating a secure and leak-free connection. The covers 202 and 222 may be made from various materials, such as stainless steel or plastic, depending on the specific application and compatibility with the sample and mobile phase being used. The body 216 may be made of various strong and clean materials, such as stainless steel, glass, ceramic, titanium, high-density polyethylene (HDPE), silicone, among others.

When the divider 120 is assembled, the internal conduits 218 may be filled with agarose or polymers as the stationary phase for SEC. For example, the stationary phase of the internal conduits 218 may include agarose, polyacrylamide, dextran, sephadex, poly styrene-divinylbenzene, or other resin composites.

For example, agarose is a polysaccharide that may be used as a stationary phase in SEC. Agarose is a highly porous material that allows separation of molecules based on size, used for the separation of proteins, nucleic acids, and polysaccharides. Polyacrylamide is a polymer that may be used as a stationary phase in gel electrophoresis and ion exchange chromatography. Polyacrylamide may be cross-linked to form a gel with controlled porosity and charge density, used for the separation of proteins and nucleic acids.

Dextran is a polysaccharide that may be used as a stationary phase in SEC. Dextran is similar to agarose in the size exclusion properties but has a different chemical structure. Dextran is used for the separation of proteins, nucleic acids, and polysaccharides. Sephadex is a cross-linked polymer that may be used as a stationary phase in gel filtration chromatography. Sephadex is similar to agarose and dextran but has a different chemical structure. Sephadex is used for the separation of proteins, nucleic acids, and polysaccharides. Poly styrene-divinylbenzene (PSD) is a cross-linked polymer that may be used as a stationary phase in size exclusion and ion exchange chromatography. PSD has a high surface area and can be modified with different functional groups to impart selectivity for different types of molecules.

The performance of the divider 120 may rely on various factors, including column efficiency (column referring to the stationary phase in the conduits), particle size of the stationary phase, mobile phase flow rate, mobile phase composition, detector sensitivity, sample volume, temperature, among others. For example, an efficient column may separate components effectively and result in high resolution analyses. The particle size of the stationary phase may determine the measurement resolution, as smaller particles may result in better separation but require an increase in backpressure and a decrease in column life. The flow rate of the mobile phase may affect the resolution by varying the time that analytes spend in the column. A slower flow rate may increase resolution by allowing more time for separation, but the slower flow rate can also increase analysis time.

When considering other factors, the composition of the mobile phase may affect resolution as different solvents or additives may improve separation by changing the selectivity of the separation. The sensitivity of the detector 130 used to detect the separated analytes may affect the resolution. A more sensitive detector can detect smaller differences in analyte concentration and improve the resolution. The volume of the sample injected into the chromatography column of the divider 120 may affect the resolution, as injecting a large amount of sample may lead to peak broadening and a decreased resolution. In addition, higher environmental temperatures can decrease the viscosity of the mobile phase and improve separation, but they can also lead to column degradation and decreased lifetime.

In view of these factors, the divider 120 allows various configuration utilizing any number of the multiple conduits 218 included in the body 216. Although FIGS. 2-5 illustrates four parallel conduit columns, the divider 120 may include any different number of conduits 218 (e.g., four or more) in the body 216, and the multiple conduits 218 may have different curvatures or shapes.

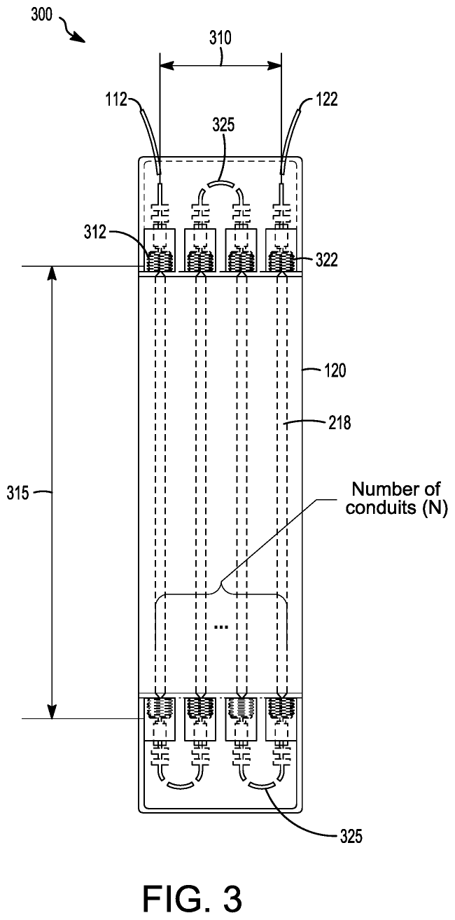

FIG. 3 illustrates an example front view 300 of the divider of FIG. 2, in accordance with certain aspects of the present disclosure. As shown, the divider 120 includes an inlet 312 and an outlet 322, which respectively provide ports for the incoming sample 112 and the outgoing eluent 122. The distance 310 between the inlet 312 and the outlet 322 is less than the length 315 of each conduit 218. In other examples, according to aspects of this disclosure, the distance 310 is less than the total conduit length of the stationary phase. For example, suppose there are N conduits (N=2, 3, 4, 5, . . . ), and suppose the connection tube length 325 is standard. The following geometrical relationship remains true according to the present disclosure:

$$Distance 310 < Conduit Length 315 \times N + Tube Length 325 \times ( N - 1 )$$

As such, the relatively short distance 310 (e.g., compared to the conduit length 315) enables practical handling of the divider 120 in medical facilities, when the divider 120 may be conveniently replaced.

In some embodiments, the conduit length 315 may be 15 cm to 25 cm. The total conduit length may be 60 cm to 100 cm, such as 80 cm. The conduits 218 may have a diameter of 2.5-15 mm, such as 5 mm.

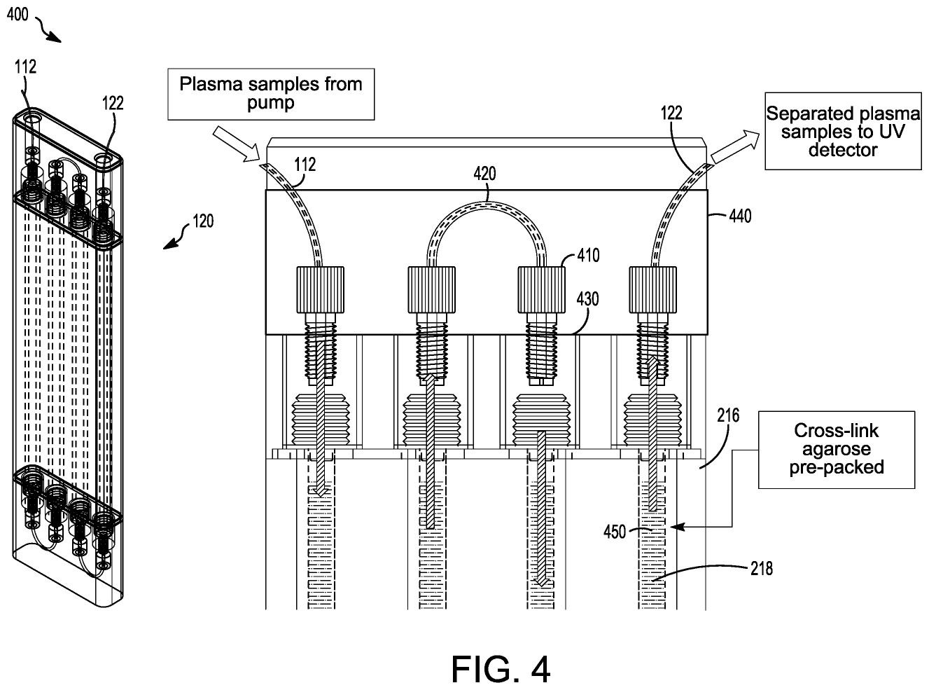

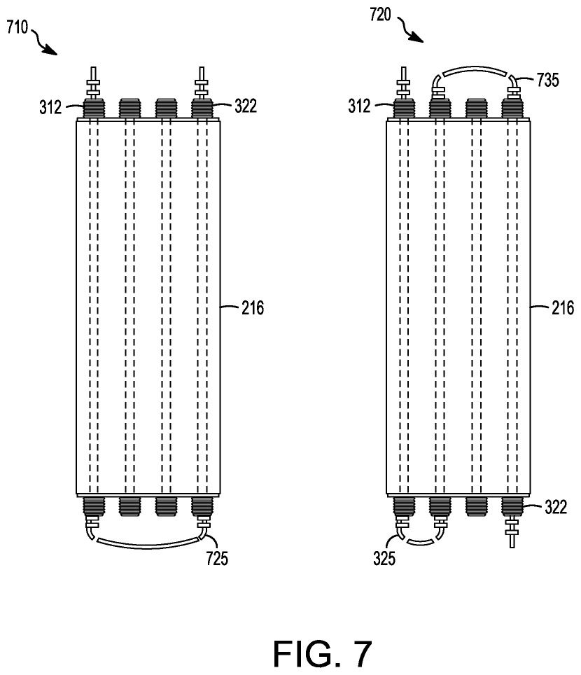

FIG. 4 illustrates an example perspective view and a local view 400 of the divider of FIG. 2, in accordance with certain aspects of the present disclosure. As shown, the conduits 218 are packed with agarose 450 in all columns. The plasma sample 112 goes through a zig-zag pathway created by the cap connectors 430, the fittings 410, and the tube 420. Details of the cap connectors 430 are illustrated in FIG. 5. Details of the fittings 410 (as well as the fitting and tube assembly 206) are illustrated in FIG. 6. In FIG. 4, the configuration 440 of the fittings 410 and tubes 420 may vary depending on the number of conduits 218, the desired total column length, and other factors. FIG. 7 illustrates two other configurations 710 and 720 on the same body 216 while changing the inlet 312 and outlet 322 locations.

In some embodiments, the agarose 450 for SEC may have a particle size of 8.6-30 microns. The sample volume may be between 50 to 200 micro-liters. For the mobile phase, the solvent may include 20 mM Tris+0.5 mM EDTA+0.02% NaN₃. The operation temperature may be between 2-40 degrees Celsius.

FIG. 5 illustrates an example cap 430, its perspective view 500 and an enlarged cross sectional view for use with the divider 120 of FIG. 2, in accordance with certain aspects of the present disclosure. As shown, the cap 430 includes external engagement surfaces 510 for receiving a torque that rotates the cap 430 with respect to the body 216. The cap 430 includes a first set of threads 522 for engaging with the threaded ports 212 on the body 216. The cap 430 further includes an O-ring 512 for preventing fluid leakage from the conduit 218 when installed. The cap 430 includes a frit 514 to prevent leakage of the filled cross-link agarose 450 in the conduit 218.

The cap 430 further includes a second set of threads 520 for engaging with the fitting 410. Although the figures illustrate the cap 430 and the fitting 410 being two separate components (as the fitting 410 may be a standard component available off-the-shelf), in some cases, the cap 430 and the fitting 410 may be integrated as a single component, thus not requiring the threaded connection based on the second set of threads 520 (while the illustrated cap 430 may further have features to be connected with the tube 420).

FIG. 6 illustrates fittings 410 and tubes 420 (of the assembly 206) for use with the divider 120 of FIG. 2, in accordance with certain aspects of the present disclosure. As shown, the fitting 410 may include threads 612 for assembly with the cap 430 via the second set of threads 520. The fitting 410 further includes a handle 610 for receiving a torque from a user. The fitting 410 may optionally include a male connector 620 for receiving the tube 420, which may engage the connector 620 by bonding, tight fitting, and other mating means. The tube 420 may be inserted into the cavity 614 formed at the top of the fitting 410. In some cases, the fitting 410 is an off-the-shelf, standard ¼-28 size fitting.

In some embodiments, the tube 420 is a flexible tube for transferring plasma or sample fluid. The tube 420 may be made of materials that are inert, non-reactive, and have low levels of leachable compounds that might interfere with the separation or detection of analytes. For example, the tube 420 may use polytetrafluoroethylene (PTFE), polyetheretherketone (PEEK), fluorinated ethylene propylene (FEP), silicone, Tygon®, and similar materials. The material selected for the tube 420 may be a highly inert material that is resistant to most solvents and acids.

The tube 420 may be affixed to the fittings 410 using various types of connectors or adapters. In addition to the examples shown in FIG. 6, other examples may include compression fittings, barbed fittings, Luer fittings, screw fittings, among others.

FIG. 7 illustrates configuration variations 710 and 720 of inlets 312, outlets 322, and conduit lengths of the divider 120 of FIG. 2, in accordance with certain aspects of the present disclosure. As shown, in the variation 710, the outer conduits 218 may be connected directly by a single tube 725, skipping the two conduits in the middle. This configuration reduces the agarose prepacked in the body 216. In the variation 720, the second and the fourth conduits 218 (counting from left to right) are connected via the tube 735 to skip the third conduit, thus utilizing three of the four conduits in the body 216. The variations 710 and 720, along with the example 200 shown in FIG. 2, illustrate variable configurations achievable with the body 216, which may be tailored to specific resolution, column length, or cost requirements.

In addition to the examples shown in FIGS. 2-5, and the variations 710 and 720 of FIG. 7, the present disclosure does not require all conduits 218 to be parallel or opened ended at the top and bottom of the body 216. For example, one or more of the conduits 218 may be connected internally without relying on external caps 430 or the fitting/tube assembly 206. That is, one or more conduits 218 may form a zig-zagging conduit inside the body 216 to provide a total length greater than either the width or the length of the body 216.

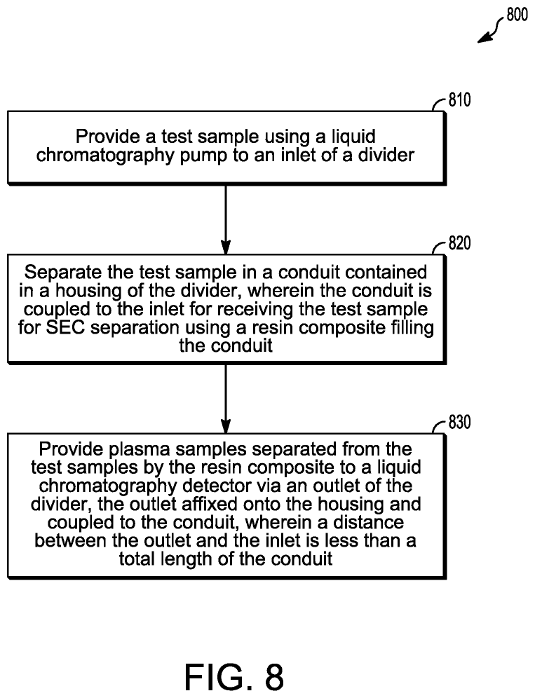

FIG. 8 illustrates a flow diagram 800 of methods of system performance using the divider 120 of FIG. 2, in accordance with certain aspects of the present disclosure. The methods in the flow diagram 800 may be performed by the system 100 shown in FIG. 1. As shown, at 810, a liquid chromatography pump provides a test sample to an inlet of a divider (such as the divider 120). At 820, the divider separates a substance from the test sample in a conduit contained in a housing of the divider. The conduit is coupled to the inlet for receiving the test sample for SEC separation using a resin composite filling the conduit. At 830, the divider provides the substance separated from the test samples by the resin composite to a liquid chromatography ultra-violet (UV) detector via an outlet of the divider. The outlet affixed onto the housing and coupled to the conduit. A distance between the outlet and the inlet is less than a total length of the conduit.

In aspects, separating the test sample in the conduit contained in the housing includes separating the test sample in a number of parallel tubes. Each of the parallel tubes may have a coupler at each end for connecting two or more of the plurality of parallel tubes in series. In some cases, the method further includes sealingly connecting an open end of a flexible tubing to a corresponding one of the plurality of parallel tubes using a fitting of the coupler. In some cases, the method further includes enclosing the coupler and the flexible tubing using a rigid cap tight-fitting onto the housing. For example, the fitting of the coupler is rotated onto the parallel tubes via an intermediate connector providing female threads for receiving male threads on the fitting of the coupler and male threads on the plurality of parallel tubes.

In some cases, the method further includes filling the conduit with the resin composite having a cross-linked agarose; and pumping plasma of the test sample through the conduit for measuring an absorbance value of a lipoprotein in the plasma using the liquid chromatography UV detector. The test sample may be the whole blood (and the plasma therein) of a patient. In some cases, the test sample may be pre-processed to remove substances irrelevant to the test, and include the plasma of concern. The plasma includes, besides the lipoprotein, a variety of proteins (such as human serum albumin (HSA)), lipids, and other molecules The lipoprotein includes high-density lipoprotein (HDL) and low-density lipoprotein (LDL). The lipoprotein also includes very low-density lipoprotein (VLDL).

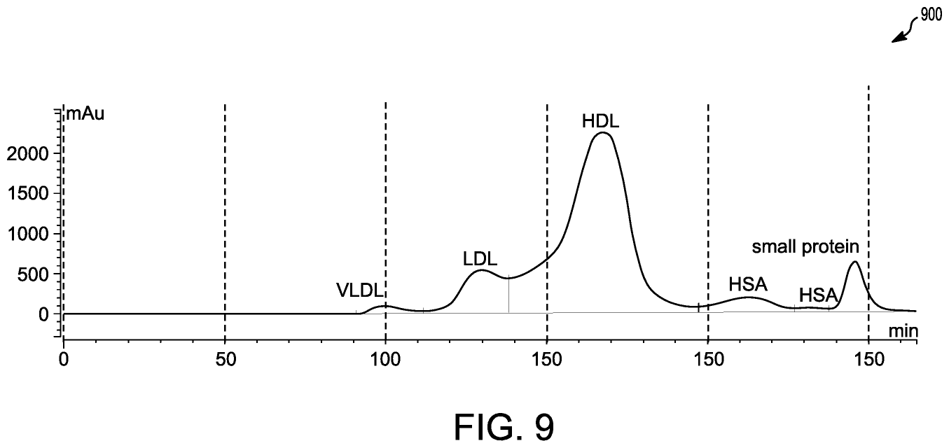

FIG. 9 illustrates example measurement results 900, in accordance with certain aspects of the present disclosure. As shown, the y-axis is the absorbance value in mAu (milli absorbance unit), and the X-axis of this chart is the residence time (in minutes), which may also be expressed as a multiple of the column volume. The value of absorbance is calculated by the following formula: Au=−log 10T %, where T % is the penetration rate. As shown in FIG. 9, the divider 120 has effectively separated VLDL, LDL, HDL, and HSA.

The preceding description sets forth numerous specific details such as examples of specific systems, components, methods, and so forth, in order to provide a good understanding of several embodiments of the present disclosure. It may be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular embodiments may vary from these exemplary details and still be contemplated to be within the scope of the present disclosure.

Additionally, some embodiments may be practiced in distributed computing environments where the machine-readable medium is stored on and or executed by more than one computer system. In addition, the information transferred between computer systems may either be pulled or pushed across the communication medium connecting the computer systems.

Embodiments of the claimed subject matter include, but are not limited to, various operations described herein. These operations may be performed by hardware components, software, firmware, or a combination thereof.

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent or alternating manner.

The above description of illustrated embodiments, including what is described in the Abstract, is not intended to be exhaustive or to limit the invention to the precise forms disclosed. While specific implementations or embodiments of, and examples for, the invention are described herein for illustrative purposes, various equivalent modifications are possible within the scope of the disclosure, as those skilled in the relevant art may recognize. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Furthermore, the terms “first,” “second,” “third,” “fourth,” etc. as used herein are meant as labels to distinguish among different elements and may not necessarily have an ordinal meaning according to their numerical designation.

It may be appreciated that variants of the above-disclosed and other features and functions, or alternatives thereof, may be combined into may other different systems or applications. Various presently unforeseen or unanticipated alternatives, modifications, variations, or improvements therein may be subsequently made by those skilled in the art which are also intended to be encompassed by the following claims. The claims may encompass embodiments in hardware, software, or a combination thereof.

What is claimed is:

1. An apparatus for substance separation using size-exclusion chromatography (SEC), the apparatus comprising:
an inlet affixed onto a housing;
a conduit contained in the housing, wherein the conduit is coupled to the inlet for receiving a test sample for SEC separation using a resin composite filling the conduit; and
an outlet affixed onto the housing and coupled to the conduit, wherein a distance between the outlet and the inlet is less than a total length of the conduit.

2. The apparatus of claim 1, wherein the conduit comprises a plurality of parallel tubes, each of the plurality of parallel tubes having a coupler at each end for connecting two or more of the plurality of parallel tubes in series.

3. The apparatus of claim 2, wherein the coupler comprises a fitting and a flexible tubing, the fitting sealingly connecting an open end of the flexible tubing to a corresponding end of the plurality of parallel tubes.

4. The apparatus of claim 3, further comprising a cap to enclose the coupler and protect the flexible tubing.

5. The apparatus of claim 4, wherein the fitting comprises a thread, a seal, and a handle for installation and removal.

6. The apparatus of claim 1, wherein the resin composite comprises a cross-linked agarose and wherein the test sample comprises plasma, such that when the plasma is pumped through the conduit, an absorbance value of a lipoprotein in the plasma is measurable.

7. The apparatus of claim 6, wherein the cross-linked agarose has a particle size between 8 and 30 microns, and wherein the test sample has a volume ranged between 50 and 200 micro-liters.

8. A system for substance separation using size-exclusion chromatography (SEC), the system comprising:
a liquid chromatography pump;
a liquid chromatography ultra-violet (UV) detector; and
a divider including:
an inlet affixed onto a housing, the inlet receiving a test sample pumped from the liquid chromatography pump;
a conduit contained in the housing, wherein the conduit is coupled to the inlet for receiving the test sample for SEC separation using a resin composite filling the conduit; and
an outlet affixed onto the housing and coupled to the conduit, wherein a distance between the outlet and the inlet is less than a total length of the conduit, and wherein the outlet provides substances separated from the test samples by the divider to the liquid chromatography UV detector.

9. The system of claim 8, wherein the conduit comprises a plurality of parallel tubes, each of the plurality of parallel tubes having a coupler at each end for connecting two or more of the plurality of parallel tubes in series.

10. The system of claim 9, wherein the coupler comprises a fitting and a flexible tubing, the fitting sealingly connecting an open end of the flexible tubing to a corresponding one of the plurality of parallel tubes.

11. The system of claim 10, further comprising a cap to enclose the coupler and protect the flexible tubing.

12. The system of claim 11, wherein the fitting comprises a thread, a seal, and a handle for installation and removal.

13. The system of claim 8, wherein the resin composite comprises a cross-linked agarose and wherein the test sample comprises plasma, such that when the plasma is pumped through the conduit, an absorbance value of a lipoprotein in the plasma is measured by the liquid chromatography UV detector.

14. The system of claim 13, wherein the cross-linked agarose has a particle size between 8 and 30 microns, and wherein the test sample has a volume ranged between 50 and 200 micro-liters.

15. A method for substance separation using size-exclusion chromatography (SEC), the method comprising:
providing a test sample using a liquid chromatography pump to an inlet of a divider;
separating a substance from the test sample in a conduit contained in a housing of the divider, wherein the conduit is coupled to the inlet for receiving the test sample for SEC separation using a resin composite filling the conduit; and
providing the substance separated from the test samples by the resin composite to a liquid chromatography ultra-violet (UV) detector via an outlet of the divider, the outlet affixed onto the housing and coupled to the conduit, wherein a distance between the outlet and the inlet is less than a total length of the conduit.

16. The method of claim 15, wherein separating the test sample in the conduit contained in the housing comprises:
separating the test sample in a plurality of parallel tubes, each of the plurality of parallel tubes having a coupler at each end for connecting two or more of the plurality of parallel tubes in series.

17. The method of claim 16, further comprising sealingly connecting an open end of a flexible tubing to a corresponding one of the plurality of parallel tubes using a fitting of the coupler.

18. The method of claim 17, further comprising enclosing the coupler and the flexible tubing using a rigid cap tight-fitting onto the housing.

19. The method of claim 18, further comprising rotating the fitting of the coupler onto the plurality of parallel tubes via an intermediate connector providing female threads for receiving male threads on the fitting of the coupler and male threads on the plurality of parallel tubes.

20. The method of claim 15, further comprising:
filling the conduit with the resin composite having a cross-linked agarose; and
pumping plasma of the test sample through the conduit for measuring an absorbance value of a lipoprotein in the substance using the liquid chromatography UV detector.
